(19) United States
(12) Patent Application Publication
(10) Pub. No.: US 2012/0225025 A1
(43) Pub. Date: Sep. 6, 2012

(54) DYE TRANSFER INHIBITING POLYMERS WITH IMPROVED STORAGE STABILITY AND PROCESSABILITY FOR LAUNDRY DETERGENTS AND CLEANERS

(75) Inventor: Frank-Peter LANG, Hattersheim (DE)

(73) Assignee: BASF SE, Ludwigshafen (DE)

(21) Appl. No.: 13/405,534

(22) Filed: Feb. 27, 2012

Related U.S. Application Data
(60) Provisional application No. 61/447,727, filed on Mar. 1, 2011.

(51) Int. Cl. A61K 8/81; A61K 8/72; A61Q 5/06; C11D 3/60

(52) U.S. Cl. 424/70.15; 510/513; 510/276; 510/353; 424/70.11; 424/70.17

(57) ABSTRACT

The present invention relates to dye transfer inhibiting polymers in powder, pellet and granule form, having higher storage stability and improved processability and to their application in laundry detergents and cleaners, in textile auxiliaries, and also further applications in cosmetics.

The present invention relates to DTI polymers in powder, pellet and granule form, having higher storage stability and improved processability and to their application in laundry detergents and cleaners, in textile auxiliaries, and also further applications in cosmetics.

DTI (dye transfer inhibiting) polymers have long been an important constituent of state of the art laundry detergents and cleaners for textiles. Their function is to bind dyes which bleed during the washing of colored textiles and so prevent their redeposition on white or differently colored textiles also present in the wash, and thereby protect the latter from tainting or staining. The problem of textile dyeings bleeding arises particularly in the case of intensively colored textiles and here more particularly from the use of direct or substantive dyes. But other classes of dyes, such as reactive dyes, can also be the cause of bleeding if, for cost reasons for example, the dyeing operation is not concluded with an adequate aftertreatment of the textile (washing off, application of dye fixatives).

Today, DTI polymers are used in all types of laundry detergents, for example color detergents, heavy-duty detergents, mild detergents, specialty detergents for black textiles, detergents for jeans, and wool detergents. The use concentration is generally between 0.1% and 2.0% by weight, particularly between 0.2% and 1% by weight and especially between 0.3% and 0.7% by weight of DTI polymer based on the laundry detergent. The laundry detergents in which DTI polymers are used can be in solid form, for example in the form of powders, granules, pellets, tablets, bar soaps, in semi-liquid form, for example in the form of gels or pastes, or in liquid form.

DTI polymers comprise homo- or copolymers based on vinylic, nitrogenous, preferably heterocyclic monomers, for example N-vinylpyrrolidone, N-vinylimidazole, N-vinylcaprolactam and 4-vinylpyridine. Certain monomers, such as 4-vinylpyridine for example, can also be present in the homo- or copolymers in derivatized form, for example as N-carboxymethyl-betaine, as N-sulfopropylbetaine or as N-oxide. Useful monomers for the synthesis of DTI polymers further include, for example, N-vinylpyridine, diallyldimethylammonium chloride, N-vinylformamide, N-vinylacetamide, vinylamine, allylamine, acrylamide and N-substituted acrylamides. In addition to the vinylic nitrogenous monomers, further nitrogen-free co-monomers can be used in order that secondary properties, such as the formulatability in liquid laundry detergent preparations for example, may be improved.

In addition to the use of the abovementioned DTI polymers in laundry detergents and cleaners, where they are used because of their ability to complex dyes bleeding out of textiles, these polymers, based on nitrogenous monomers, additionally find use in other applications. For instance, polyvinylpyrrolidone and copolymers of vinylpyrrolidone with other monomers such as vinylimidazole, vinyl acetate or diallyldimethylammonium chloride are used in the cosmetics industry to formulate hairspray and hair styling products. Polydimethyldiallyammonium chloride is used inter alia in hair rinses on account of its conditioning effect and to improve combability.

To use the DTI polymers in laundry detergent and cleaner compositions, as well as in other fields of use, they are on offer as aqueous solutions or in solid form, as powders or granules.

Aqueous solutions of DTI polymers have the disadvantage of only being obtainable with relatively low active-ingredient contents, which increases the cost of transportation to the customer. They further require heatable tanks for storage and corresponding systems to feed them to the manufacturing or processing operation, for example pumps, pipework and nozzles for spraying onto for example the washing powder matrix, which represents an appreciable capital expense.

The powder-, pellet- and granule-shaped DTI polymers that are commercially available in turn have the disadvantage of being very hygroscopic. This greatly inconveniences their handling in manufacture, particularly in climatic zones having a comparatively high humidity. Therefore, it is often the case that controlled climatic conditions have to be taken, which again entails an appreciable capital expense. Moreover, opened containers have to be carefully resealed, which can be a problem with Big Bags. Irrespective of their hygroscopicity, particularly the pulverulent DTI polymers exhibit high forces of cohesion between the particles and hence also very poor flow performance, which appreciably inconveniences conveying and exact metering. Finally, the products have poor storage stability when exposed to the mechanical impact of pressure which acts on the packaged products in the stacking of Big Bags for example and which leads to caking.

It is an object of the present invention to provide a system in which the DTI polymers are present in the form of powders, granules or pellets, these have good physical stability and are simple to store and process by the user.

We have found that this object is achieved, surprisingly, when certain finely divided additives are added to the DTI polymers which are present in solid form. The present invention accordingly provides a mixture comprising DTI polymer or polymers, preferably powder-, granule- or pellet-shaped, and one or more, preferably finely divided, additive(s). In this case, a mixture consisting of a DTI polymer in powder, pellet and/or granule form and a finely divided additive is a particularly preferred embodiment.

This particularly preferred embodiment will now be described, but all other embodiments, i.e., with multiple polymers and/or multiple additives, are similarly improvable via further features and hence also described mutatis mutandis.

The mixture of the present invention comprises the DTI polymer in solid form, i.e., in the form of powders, granules or pellets, in an amount of 90% to 99.99% by weight, and also 0.01% to 10% by weight of a finely divided additive, the weight %ages being based on the total amount of the two constituents. In a preferred embodiment, the mixture consists of DTI polymer and finely divided additive. For the purposes of this invention, powder-shaped materials have a particle size in the range from 1 μm to 0.1 mm, granule-shaped materials have a particle size in the range from 0.1 mm to 2 mm and pellet-shaped particles have a particle size in the range from 2 mm to 5 mm.

The DTI polymer, as mentioned, may be constructed on the basis of nitrogenous monomers polymerizable via vinyl or allyl groups. Examples thereof are N-vinylpyrrolidone, N-vinyl-imidazole, N-vinylcaprolactam, 4-vinylpyridine, N-vinylpyridine, diallyldimethylammonium chloride, N-vinylformamide, N-vinylacetamide, vinylamine, allylamine, acrylamide, N-substituted and also N,N-substituted acrylamides. The nitrogen atoms in the homo- or copolymers may also be present therein in derivatized form. This derivatization is preferably carried out after the polymerization and comprises for example quaternizations, for example with methyl chloride, benzyl chloride, dimethyl sulfate; betainizations for example with chloracetic acid, propanesultone or oxidations for example with hydrogen peroxide. An example thereof is poly(4-vinylpyridine) which is used as a DTI polymer following derivatization in the form of the N-carboxymethylbetaine, the N-sulfopropylbetaine or the N-oxide.

The copolymers may comprise units derived from further nitrogen-free monomers. Examples thereof are vinyl acetate (which after polymerization may be completely or partially hydrolyzed to the vinyl alcohol unit); C2-C24 olefins, preferably ethylene, propylene, butylene; diisobutene; vinylbenzene (styrene); vinyl chloride; acrylic acid and also its esters with aliphatic, alicyclic, aromatic alcohols R—OH where R═C1 to C18-alkyl, benzyl, ethylphenyl, cyclohexyl or phenyl, preferably with methanol, ethanol or propanol, polyethylene glycols (PEGs) and one-sidedly endblocked polyethylene glycols (methyl polyethylene glycols, MPEGs); methacrylic acid, and also its esters with aliphatic, alicyclic or aromatic alcohols R—OH where R═C1- to C18-alkyl, benzyl, ethylphenyl, cyclohexyl or phenyl, preferably with methanol, ethanol or propanol, polyethylene glycols (PEGs) and one-sidedly endblocked polyethylene glycols (methyl polyethylene glycols, MPEGs); amides of acrylic acid or of methacrylic acid with primary or secondary amines which may have aliphatic (C1- to C22-alkyl), alicyclic (e.g., hexyl) or aromatic (e.g., phenyl, benzyl) substituents; vinylsulfonic acid; allylsulfonic acid; 2-acrylamido-2-methylpropylsulfonic acid (AMPS).

Examples of DTI polymers include polyvinylpyrrolidone (e.g., Sokalan® HP 50/BASF, PVP-K-Typen®/ISP), vinylpyrrolidone-vinylimidazole copolymer (e.g., Sokalan® HP 56/BASF), poly(4-vinylpyridin N-oxide) (e.g., Chromabond® S-403E /ISP), poly(4-vinylpyridine N-carboxymethyl-betaine (e.g., Chromabond® S 400/ISP).

Further DTI polymers are polymers having aminal, hemiaminal and aminal-acetal structures as described in DE 44 13 720 and copolymers of vinylamido monomers and vinyl ester monomers as described in EP 0 753 566.

Useful DTI polymers further include polycationic condensates which are obtainable by reaction of piperazine, 1-alkylpiperazine, 1,4-dialkylpiperazine, 1,4-bis(3-aminopropyl)piperazine, 1-(2-aminoethyl)piperazine, 1-(2-hydroxyalkyl)piperazine, imidazole, alkylimidazole with alkylene dihalides, epihalohydrin and/or bisepoxides and which can subsequently be quaternized with alkylating agents as described in EP 0 934 382.

Polymers that differ in the monomers used or in the ratio between the monomers used can also be used as a mixture in the present invention mixtures of DTI polymers and additive.

Suitable additives are for example fatty acids, particularly C₁₆-C₂₂ fatty acids, such as tallow fatty acid, stearic acid, behenic acid and salts thereof, preferably alkaline earth metal ions, more preferably with Ca²⁺ and Mg²⁺; fatty alcohols; cellulose; waxes, for example montan waxes, paraffin waxes, ester waxes and polyolefin waxes; magnesium oxide; kaolin; talc, tricalcium phosphate and silicas.

The aforementioned additives can be used not only individually but also in admixture in the mixture of the present invention, comprising DTI polymer(s) and additive(s).

Preferred additives include the Ca²⁺ and Mg²⁺ salts of C₁₆-C₂₂ fatty acids, especially calcium stearate and magnesium stearate. Particularly preferred additives are synthetic, colloidal, pyrogenous silicas and synthetic, colloidal, precipitated silicas. Pyrogenous silicas are obtained by high-temperature flame hydrolysis of silicon tetrachloride in the detonating gas flame. Precipitated silicas are obtained wet-chemically from alkali metal silicate solutions by addition of acids. Pyrogenous silicas as well as precipitated silicas are amorphous in structure, not crystalline.

Examples of pyrogenous silicas are the Aerosil® brands (from Evonik), particularly Aerosil® 200, and examples of the precipitated silicas are the Sipernat® brands (from Evonik), particularly Sipernat® 320, Sipernat® 320 DS, Sipernat® 360, Sipernat® 500 LS, Sipernat® 2200, Sipernat® 22, Sipernat® 22 S, Sipernat® 22 LS, Sipernat® 50, Sipernat® 50 S, Sipernat® C 600, Sipernat® C 630, Sipernat® 820 A and Sipernat® 880. The present invention mixtures of DTI polymer and additive can utilize not only the aforementioned hydrophilic silicas but also hydrophobically modified silicas. Hydrophobic silicas are for example Sipernat® D 10, Sipernat® D 17 and Aerosil® R 812 and R 972. Hydrophobically modified, colloidal, synthetic precipitated silicas and hydrophobically modified, colloidal, pyrogenously produced silicas are very particularly preferred additives.

Colloidal, synthetic precipitated silicas are characterized by a high specific surface area of 30-500 m²/g, preferably 150-450 m²/g. Hydrophobically modified precipitated silicas have a high specific surface area of preferably 75-125 m²/g. (Determination as per area meter method ISO 5794-1 Annex D). The tamped density is 50-300 g/L, preferably 75-200 g/L and even more preferably 90-150 g/L. (Determined to DIN ISO 787/11 after tamping under defined conditions). The colloidal pyrogenous silicas preferably have specific surface areas of 100-400 m²/g and particle sizes of 1 nm-50 nm. Tamped density is about 50-150 g/L.

The synthetic silicas described, in contradistinction to the silicates used in laundry detergents, comprise metal ions such as Ca²⁺, Mg²⁺, Al³⁺ or Fe³⁺ in very low amounts, i.e., <5% by weight, preferably less than 3% by weight, more preferably less than 2% by weight and more preferably less than 1.5% by weight, based on the total weight of the particular synthetic silica. These do not constitute a requisite or suitable constituent part of the chemical compound, but are impurities introduced by the nature of the manufacturing operation. For instance, the sodium content determined as Na₂O (ISO 3262-18) is <1.5% by weight. Crystalline silicates, sheet- silicates and zeolites do not comprise part of the preferred additives.

The present invention mixtures comprising the DTI polymer comprise the aforementioned additives in a concentration of 0.01% to 10% by weight, preferably 0.1% to 5% by weight, more preferably 0.2% to 3% by weight and most preferably 0.5% to 2% by weight.

The mixtures of the present invention are produced by mixing the DTI polymer, present as a powder, pellet or granules, with the finely divided additive.

The typical particle size of the DTI polymer is in the range from 10 to 2500 μm, preferably in the range from 20 to 1500 μm, more preferably in the range from 50 to 1000 μm and most preferably in the range from 100 to 700 μm.

By “finely divided” in reference to the additive is meant a particle size in the range from 1 to 500 μm.

In the case of the colloidal pyrogenous silicas the additives preferably have a size in the range from 1 to 50 nm. In the case of the precipitated silicas the additives preferably have a size in the range from 1 to 200 μm preferably in the range from 5 to 150 μm and more preferably in the range from 8 to 120 μm. Each determined by light scattering as per ISO13320-1.

The mixing operation can be carried out in the customary mixing assemblies, for example drum mixers, V-blenders, tumble or Turbula mixers, cone mixers (e.g., Nauta mixers), plowshare mixers (Lödige mixer, Eirich mixer). In one preferred embodiment, the mixing operation takes place in mixers that exert low shearing forces on the material being mixed, for example tumble mixers, cone mixers and plowshare mixers.

Typically, the DTI polymer is initially charged, then the additive is added and this is followed by mixing. In order to ensure gentle mixing, the shortest possible mixing times are employed. For example, a mixing time of 3 minutes is completely sufficient to produce 100 g of the mixture of the present invention in a Turbula mixer.

When the DTI polymers in powder form are produced via spray drying, the additive is advantageously metered directly to the spray tower separately from the aqueous solution of the polymer. Possible points for addition are the upper end of the spray dryer, a metering terminal via a sight glass or the metered addition via the hot air stream. The same holds in principle for spray granulation.

When metered addition of the additive into the spray tower is not possible, it can be added in a separate mixing assembly, as previously described.

Should the additive to be admixed to the DTI polymer not be sufficiently finely divided because, for example, it is only available as a material which has been melted by heating and has resolidified after filling into drums, as may be the case with fatty acids, fatty alcohols or waxes for example, the DTI polymer can be ground together with the additive, likewise ensuring commixing of the components. Any grinding apparatus is suitable for this in principle, such as impact mills and cutting mills for example.

The use concentration of the present invention mixture comprising DTI polymer and additive in laundry detergents and cleaners depends on the concentration of the DTI polymer present in the mixture. Based on the polymer (without additive) the use concentration in the laundry detergent is between 0.1% and 2.0% by weight, preferably between 0.2% and 1% by weight and most preferably between 0.3% and 0.7% by weight.

The laundry detergent and cleaner formulations in which the present invention mixtures comprising DTI polymer and additive can be used are preferably solid laundry detergents in the form of powders, granules, pellets, tablets or bar soaps. However, they can also be incorporated into liquid laundry detergents, laundering gels and laundering pastes. This will preferably be the case when long transists to the user make it desirable to supply the DTI polymer in a highly concentrated form and hence not as an aqueous solution, and the protection of the product from caking due to moisture absorption in the course of transportation (on a ship for example) is to be improved.

Laundry detergents and cleaners comprising the present invention mixture comprising DTI polymer and additive can further comprise further customary constituents. These will now be described:

Anionic Surfactants

The anionic surfactants used are preferably alkylbenzenesulfonates, alkanesulfonates, olefinsulfonates, alkyl ester sulfonates, alkyl sulfates, alkyl ether sulfates alkyl carboxylates (soaps) and alkyl phosphates. The counter-ions present are alkali metal cations, preferably sodium or potassium, alkaline earth metal cations, for example calcium or magnesium, and also ammonium NH₄+ and substituted ammonium compounds, for example mono-, di- or triethanolammonium cations, and mixtures of the aforementioned cations therefrom.

Alkenyl- or alkylbenzenesulfonates may comprise a branched or linear, optionally hydroxyl-substituted alkenyl or alkyl group. Preferably they comprise linear alkyl chains having 9 to 25 carbon atoms and more preferably having 10 to about 13 carbon atoms.

Alkanesulfonates are available on a large industrial scale in the form of secondary alkanesulfonates wherein the sulfo group is attached to a secondary carbon atom of the alkyl moiety. The alkyl group can in principle be saturated, unsaturated, branched or linear and optionally hydroxyl substituted. Preferred secondary alkanesulfonates comprise linear C₉ to C25-alkyl radicals, preferably C₁₀ to C₂₀-alkyl radicals and more preferably C₁₃ to C₁₇-alkyl radicals.

Olefinsulfonates are obtained by sulfonation of C₈ to C₂₄— and preferably C₁₄ to C₁₆-α-olefins with sulfur trioxide and subsequent neutralization. Owing to their production process, these olefinsulfonates may comprise minor amounts of hydroxyalkanesulfonates and alkanedisulfonates.

Alkyl ester sulfonates derive for example from linear esters of C₈ to C₂₀-carboxylic acids, i.e., fatty acids, which are sulfonated with sulfur trioxide. The fatty acids are obtained from natural fats, such as tallow, coconut oil and palm oil for example, or can be synthetic in nature. Compounds of formula (1)

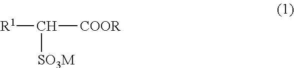

are preferred for laundry detergent and cleaner applications.

In formula (1), R¹ is a C₈ to C₂₀-alkyl radical, preferably C₁₀ to C₁₆-alkyl and R is a C₁ to C₆-alkyl radical, preferably a methyl, ethyl or isopropyl group. Particular preference is given to methyl ester sulfonates where R¹ is C₁₀ to C₁₆-alkyl.

Alkyl sulfates are surfactants of the formula ROSO₃M, where R is C₁₀-C₂₄-alkyl and preferably C₁₂-C₁₈-alkyl. M is a counter-ion as described at the beginning for anionic surfactants.

Alkyl ether sulfates have the general structure RO(A)ₘ SO₃M, where R is a C₁₀-C₂₄-alkyl and preferably C₁₂-C₁₈ alkyl radical.

A is an alkoxy unit, preferably ethoxy and m is a value from about 0.5 to about 6, preferably between about 1 and about 3, and M is a cation, for example sodium, potassium, calcium, magnesium, ammonium or a substituted ammonium cation.

Alkyl carboxylates are generally known by the term “soap”. Soaps can be manufactured on the basis of saturated or unsaturated, preferably native, linear C₈ to C₁₈-fatty acids.

Further anionic surfactants are salts of acylamino carboxylic acids, acyl sarcosinates, fatty acid-protein condensation products obtained by reaction of fatty acid chlorides with oligopeptides; salts of alkylsulfamido carboxylic acids; salts of alkyl and alkylaryl ether carboxylic acids; sulfonated polycarboxylic acids, alkyl and alkenyl glycerol sulfates such as oleyl glycerol sulfates, alkylphenol ether sulfates, alkyl phosphates, alkyl ether phosphates, isethionates, such as acyl isethionates, N-acyltaurides, alkyl succinates, sulfosuccinates, monoesters of sulfosuccinates (particularly saturated and unsaturated C₁₂-C₁₈-monoesters) and diesters of sulfosuccinates (particularly saturated and unsaturated C₁₂-C₁₈-diesters), sulfates of alkylpolysaccharides such as sulfates of alkylpolyglycosides and alkyl polyethoxy carboxylates such as those of the formula RO(CH₂CH₂)ₖCH₂COO⁻M⁺, where R is C₈ to C₂₂-alkyl, k is a number from 0 to 10 and M is a cation.

Nonionic Surfactants

A distinction is made according to the type of the hydrophobic and the hydrophilic base:

Condensation products of alcohols with ethylene oxide. The alcohols have a C₈ to C₂₂-alkyl group, preferably a C₁₀ to C₁₈-alkyl group, which may be linear or branched, primary or secondary. These are condensed with about 1 mol to about 25 mol and preferably with about 3 mol to about 18 mol of ethylene oxide per mole of alcohol. Examples of commercially available nonionic surfactants of this type are the Lutensol brands (BASF), the Empilan brands (Huntsman) and the Genapol brands (Clariant).

Condensation products of alcohols with ethylene oxide and a further alkylene oxide. These can be constructed according to the scheme R—O-EO-AO or R—O-AO-EO, where R is a primary or secondary, branched or linear C₈ to C₂₂-alkyl group, preferably a C₁₀ to C₁₈-alkyl group, EO is ethylene oxide and AO comprises an alkylene oxide, preferably propylene oxide, butylene oxide or pentylene oxide. Known products are the Plurafac LF brands (BASF).

Condensation products of polypropylene glycol with ethylene oxide.

The hydrophobic moiety of these compounds preferably has a molecular weight between about 1500 and about 1800. The addition of up to about 40 mol of ethylene oxide onto this hydrophobic moiety leads to amphiphilic compounds. Commercially available examples of this class of products are the Pluronic® brands from BASF and the Genapol® PF brands from Clariant GmbH.

Condensation products of ethylene oxide with a reaction product of propylene oxide and ethylenediamine.

The hydrophobic unit of these compounds consists of the reaction product of ethylenediamine and propylene oxide and generally has a molecular weight of about 2500 to 3000. It is onto this hydrophobic unit that ethylene oxide is added up to a content of about 40% to about 80% by weight of polyoxyethylene and a molecular weight of about 5000 to 11 000. Commercially available examples of this class of compounds are the Tetronic® brands (BASF) and the Genapol® PN brands (Clariant).

Fatty Acid Amides

Fatty Acid Amides have the General Formula

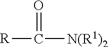

where R is an alkyl radical having 7 to 21 and preferably 9 to 17 carbon atoms.

The two R¹ alkyl radicals may be the same or different and may independently be hydrogen, C₁l-C₄-alkyl, C₁-C₄-hydroxyalkyl or (C₂H₄O)ₓH, where x varies from 1 to 3. Preference is given to C₈-C₂₀-amides as monoethanolamides, diethanolamides and diisopropanolamides.

Semipolar Nonionic Surfactants

Nonionic surfactants also include water-soluble amine oxides, water-soluble phosphine oxides and water-soluble sulfoxides, having at least one C8 to C18-alkyl radical, preferably a C10 to C14-alkyl radical which confers the amphiphilic character on the compound. Preferably C₁₀-C₁₈-alkyldimethylamine oxides and C₈-C₁₂-alkoxyethyldihydroxyethylamine oxides are used in laundry detergents and cleaners.

Useful nonionic surfactants further include alkyl and alkenyl oligoglycosides and also fatty acid polyglycol esters or fatty amine polyglycol esters each having 8 to 20 and preferably 12 to 18 carbon atoms in the fatty alkyl moiety, and fatty acid N-alkylglucamides. Zwitterionic Surfactants

Typical examples of amphoteric or zwitterionic surfactants are alkylbetaines, alkylamide betaines, aminopropionates, aminoglycinates and amphoteric imidazolinium compounds. Particular preference is given to N-alkyl-N,N-dimethyl-N-carboxymethylbetaines and also N-(alkylamidopropyl)-N,N-dimethyl-N-carboxymethylbetaines, and also alkyldipolyethoxybetaines, each with a linear or branched alkyl radical of 8 to 22 carbon atoms, preferably 8 to 18 carbon atoms and more preferably having about 12 to about 18 carbon atoms.

Cationic Surfactants

These are substituted or unsubstituted straight-chain or branched quaternary ammonium salts of the type R¹N(CH₃)₃⁺X⁻, R¹R²N(CH₃)₂⁺X⁻, R¹R²R³N(CH₃)⁺X⁻or R¹R²R³R⁴N⁺X⁻. The R¹, R², R³ and R⁴ radicals may preferably be independently unsubstituted alkyl having a chain length between 8 and 24 carbon atoms and more particularly between 10 and 18 carbon atoms, hydroxyalkyl having about 1 to about 4 carbon atoms, phenyl, C₂- to C₁₈-alkenyl, C₇- to C₂₄-aralkyl, (C₂H₄O)ₓH, where x is from about 1 to about 3, alkyl radicals comprising one or more ester groups, or cyclic quaternary ammonium salts. X is a suitable anion.

Builders

Builders can be present in the laundry detergent and cleaner compositions at weight fractions of about 5% to about 80%. Builders comprise for example alkali metal, ammonium and alkanolammonium salts of polyphosphates such as, for example, tripolyphosphates, pyrophosphates, glassy polymeric metaphosphates, orthophosphates, phosphonates, silicates, carbonates including bicarbonates and sesquicarbonates, sulfates and aluminosilicates.

Aluminosilicate builders are preferred for the present invention. Particular preference is given to zeolites of the formula Na_{z}[(AlO₂)_{z}(SiO₂)_{y}].xH₂O, where z and y are integers of at least 6, the ratio of z to y is between 1.0 to about 0.5, and x is an integer from about 15 to about 264. These aluminosilicates can be crystalline or amorphous in structure, and can be naturally occurring or else synthetically produced. Processes for preparing ion exchangers based on aluminosilicate are described in U.S. Pat. No. 3,985,669 and U.S. Pat. No. 4,605,509. Particular preference is given to synthetic crystalline aluminosilicates, such as zeolite A, zeolite P(B) and zeolite X.

Alkali metal silicates, particularly those having an SiO₂:Na₂O ratio between 1.6:1 and 3.2:1 and also sheet-silicates, for example sodium sheet-silicates as described in U.S. Pat. No. 4,664,839, e.g., SKS 6® (Clariant).

Bleaching Agents

The laundry detergent and cleaner compositions comprising the present invention preparations of dye transfer inhibitors may further comprise one or more bleaching agents and also bleach activators, bleach catalysts and suitable stabilizers. The bleaching agents used are persalts such as perborates (perborate monohydrate, perborate tetrahydrate) and percarbonate. Persalts are generally combined with so-called bleach activators, preferably tetraacetylethylenediamine. Bleach activators are often referred to in the prior art as organic peroxyacid precursors since they react with the persalt to liberate a peracid such as peracetic acid for example. Depending on the stability of the peracid in question, its physical state and the form of laundry detergent, it can also be used directly in the laundry detergent. Examples of peroxyacids preferred for use in this invention include peroxydodecanedioic acid (DPDA), the nonylamide of peroxysuccinic acid (NAPSA), the nonylamide of peroxyadipic acid (NAPAA) and decyldiperoxysuccinic acid (DDPSA), nonanoylamidocaproyloxybenzenesulfonic acid and alkanoyloxybenzenesulfonic acids such as nonanoyloxybenzenesulfonic acid (NOBS) and lauroyloxybenzenesulfonic acid (LOBS). Particular preference is given to using bleach systems based on perborate and/or percarbonate with the bleach activator tetraacetylethylenediamine (TAED) in the laundry detergent and cleaner compositions of the present invention.

The laundry detergent and cleaner compositions comprising the present invention mixtures comprising DTI polymer(s) and additive(s) may further comprise the customary assistants which boost the cleaning performance, serve to recondition the textile to be washed, or modify the performance characteristics of the laundry detergent composition. Suitable auxiliaries include for example enzymes, more particularly proteases, lipases, cellulases and amylases, mannanases, glycosidases, enzyme stabilizers, foam boosters, foam retarders, anti-tarnish and/or -corrosion agents, suspendants, dyes, fillers, optical brighteners, disinfectants, complexing agents, alkalis, hydrotropic compounds, antioxidants, perfumes, solvents, solubilizers, soil antiredeposition agents, dispersants, processing aids, plasticizers, softeners and antistats.

Cosmetic preparations utilize the present invention mixtures comprising DTI polymer(s) and additive(s) in formulations with the following other constituents for example, although from a performance point of view the primary focus is on other properties of the polymers, for example film formation and bonding strength (hairsprays) or improved combability (hair rinses). The following may be mentioned by way of example: PEG-40 hydrogenated castor oil, PEG/PPG-18/18 Dimethicon, Polyquaternium-46, Panthenol, Acrylate/C10-30Alkyl Acrylate CrossPolymer, PEG-25 PABA, propylene glycol, PEG-12 Dimethicon, vinylpyrrolidone-vinyl acetate (VP-VA) copolymer, dimethyl ether, decylglucoside, sodium laurylsulfate, sodium lauryl ether sulfate, cocamidopropylbetaine, citric acid, Laureth-3, sodium chloride, Polysorbate 20, PEG-150 Distearate, phytantriole, climbazole, EDTA and perfume.

### EXAMPLES

### Example 1

Mixtures of Sokalan® HP 50 (=PVP powder) with an addition of in each case 2% by weight, 3% by weight and 5% by weight of the hydrophobically modified silica Sipernat® D 17 were prepared. To this end, the requisite amounts of the two components were gently mixed in a container for 3 min using a tumble mixer. To investigate storage stability, the mixtures were stored in a Petri dish in a conditioning cabinet at 38° C./78% relative humidity and visually assessed for 4 weeks. For comparison, Sokalan® HP 50 was stored without added silica.

TABLE 1
storage test of Sokalan ® HP 50 (=PVP powder) with and without addition of
Sipernat ® D 17. Visual assessment at 38° C./78% relative humidity.

| Sokalan ® | Visual assessment at 38° C./78% relative humidity | | | | | | | |
|---|---|---|---|---|---|---|---|---|
| HP 50 | 0 h | 8 h | 24 h | 2 days | 7 days | 2 weeks | 3 weeks | 4 weeks |
| a) no | very | surface | homo- | clear, | clear, | storage | — | — |
| additive | badly | pasty | geneous | pale | pale | test | | |
| | flowable | and | cloudy, | yellow, | yellow, | ended | | |
| | powder | tacky | pasty | tacky | tacky | | | |
| | | | mass | mass | mass | | | |
| b) with | readily | flowable | flowable | flowable | flowable | flowable | flowable | flowable |
| 2.0% of | flowable | powder | powder | powder | powder | powder | powder | powder |
| Sipernat ® | powder | | | | | | | |
| D 17 | | | | | | | | |
| c) with | readily | readily | readily | readily | readily | readily | readily | readily |
| 3.0% of | flowable | flowable | flowable | flowable | flowable | flowable | flowable | flowable |
| Sipernat ® | powder | powder | powder | powder | powder | powder | powder | powder |
| D 17 | | | | | | | | |
| d) with | very | very | very | very | very | very | very | very |
| 5.0% of | readily | readily | readily | readily | readily | readily | readily | readily |
| Sipernat ® | flowable | flowable | flowable | flowable | flowable | flowable | flowable | flowable |
| D 17 | powder | powder | powder | powder | powder | powder | powder | powder |

Sokalan® HP 50 without addition of the additive is a poorly flowable powder which at 38° C./78% rh deliquesces after just 8 hours. The addition of just 2% of the additive leads to a distinct improvement in flowability and the product is still stable after 4 weeks (unchanged flowability at time of storage discontinuation).

### Example 2

Mixtures of Sokalan® HP 56 (=PVPVI granules) with an addition of in each case 2% by weight, 3% by weight and 5% by weight of the hydrophobically modified silica Sipernat® D 17 were prepared. To this end, the requisite amounts of the two components were gently mixed in a container for 3 min using a tumble mixer. To investigate storage stability, the mixtures were stored in a Petri dish in a conditioning cabinet at 38° C./78% relative humidity and visually assessed. For comparison, Sokalan® HP 56 was stored without added silica.

TABLE 2
storage test of Sokalan ® HP 56 with and without addition of Sipernat ® D 17. Visual
assessment at 38° C./78% relative humidity.

| Sokalan ® | Visual assessment at 38° C./78% relative humidity | | | | | | | |
|---|---|---|---|---|---|---|---|---|
| HP 56 | 0 h | 8 h | 24 h | 2 days | 7 days | 2 weeks | 3 weeks | 4 weeks |
| a) no | very | granules | yellowish | yellowish | storage | — | — | — |
| additive | readily | start to | beige | beige | test | | | |
| | flowable | stick | homo- | clear film | terminated | | | |
| | granules | together | geneous | | | | | |
| | | | mass | | | | | |
| b) with | very | very | very | very | very | very | very | very |
| 2.0% of | readily | readily | readily | readily | readily | readily | readily | readily |
| Sipernat ® | flowable | flowable | flowable | flowable | flowable | flowable | flowable | flowable |
| D 17 | granules | granules | granules | granules | granules | granules | granules | granules |
| c) with | very | very | very | very | very | very | very | very |
| 3.0% of | readily | readily | readily | readily | readily | readily | readily | readily |
| Sipernat ® | flowable | flowable | flowable | flowable | flowable | flowable | flowable | flowable |
| D 17 | granules | granules | granules | granules | granules | granules | granules | granules |
| d) with | very | very | very | very | very | very | very | very |
| 5.0% of | readily | readily | readily | readily | readily | readily | readily | readily |
| Sipernat ® | flowable | flowable | flowable | flowable | flowable | flowable | flowable | flowable |
| D 17 | granules | granules | granules | granules | granules | granules | granules | granules |

Sokalan® HP 56 without addition of the additive is very readily flowable granules which at 38° C./78% rh deliquesces after just 24 hours. The addition of just 2% of the additive leads to the granules exhibiting a significantly improved storage stability of more than 4 weeks (unchanged at time of storage discontinuation).

### Example 3

Mixtures of Sokalan® HP 50 (=PVP powder) with an addition of 2% by weight of the hydrophobically modified silica Sipernat D 10 were prepared. To this end, the requisite amounts of the two components were gently mixed in a container for 3 min using a tumble mixer. To investigate storage stability, the mixtures were stored in a Petri dish in a conditioning cabinet at 38° C./78% relative humidity and visually assessed for 4 weeks. For comparison, Sokalan® HP 50 was stored without added silica.

TABLE 3
storage test of Sokalan ® HP 50 (=PVP powder) with and without addition of
Sipernat ® D 10. Visual assessment at 38° C./78% relative humidity.

| Sokalan ® | Visual assessment at 38° C./78% relative humidity | | | | | | | |
|---|---|---|---|---|---|---|---|---|
| HP 50 | 0 h | 8 h | 24 h | 2 days | 7 days | 2 weeks | 3 weeks | 4 weeks |
| a) no | very poorly | surface | homo- | clear, | clear, | storage | — | — |
| additive | flowable | pasty | geneous | bright | bright | test | | |
| | powder | and | cloudy, | yellow, | yellow, | terminated | | |
| | | tacky | pasty | tacky | tacky | | | |
| | | | mass | mass | mass | | | |
| b) with | readily | flowable | flowable | flowable | flowable | flowable | flowable | flowable |
| 2.0% of | flowable | powder | powder | powder | powder | powder | powder | powder |
| Sipernat ® | powder | | | | | | | |
| D 10 | | | | | | | | |

### Example 4

Mixtures of Sokalan® HP 56 (=PVPVI granules) with an addition of 2% by weight of the hydrophobically modified silica Sipernat D 10 were prepared. To this end, the requisite amounts of the two components were gently mixed in a container for 3 min using a tumble mixer. To investigate storage stability, the mixtures were stored in a Petri dish in a conditioning cabinet at 38° C./78% relative humidity and visually assessed. For comparison, Sokalan® HP 56 was stored without added silica.

TABLE 4
storage test of Sokalan ® HP 56 with and without addition of Sipernat D 10. Visual
assessment at 38° C./78% relative humidity.

| Sokalan ® | Visual assessment at 38° C./78% relative humidity | | | | | | | |
|---|---|---|---|---|---|---|---|---|
| HP 56 | 0 h | 8 h | 24 h | 2 days | 7 days | 2 weeks | 3 weeks | 4 weeks |
| a) no | very | granules | yellowish | yellowish | storage | — | — | — |
| additive | readily | start to | beige | beige, | test | | | |
| | flowable | stick | homo- | clear film | terminated | | | |
| | granules | together | geneous | | | | | |
| | | | mass | | | | | |
| b) with 2.0% | very | very | very | very | very | very | very | very |
| of Sipernat ® | readily | readily | readily | readily | readily | readily | readily | readily |
| D 10 | flowable | flowable | flowable | flowable | flowable | flowable | flowable | flowable |
| | granules | granules | granules | granules | granules | granules | granules | granules |

### Example 5

Mixtures of Sokalan® HP 56 (=PVPVI granules) with an addition of 1% by weight of magnesium stearate were prepared. To this end, the requisite amounts of the two components were gently mixed in a container for 3 min using a tumble mixer. To investigate storage stability, the mixtures were stored in a Petri dish in a conditioning cabinet at 33° C./65% relative humidity and visually assessed. For comparison, Sokalan® HP 56 was stored without added magnesium stearate.

TABLE 5
storage test of Sokalan ® HP 56 with and without addition of magnesium stearate.
Visual assessment at 33° C./65% relative humidity.

| Sokalan ® | Visual assessment at 33° C./65% relative humidity | | | | |
|---|---|---|---|---|---|
| HP 56 | 0 h | 8 h | 24 h | 2 days | 7 days |
| a) no | very | granules | yellowish | yellowish beige, | — |
| additive | readily | start to | beige | clear film | |
| | flowable | stick | homo- | | |
| | granules | together | geneous | | |
| | | | mass | | |
| b) with 1.0% | very | readily | readily | readily flowable | readily flowable |
| of | readily | flowable | flowable | granules but | granules but |
| magnesium | flowable | granules | granules | yellow | yellow |
| stearate | granules | | | discoloration | discoloration |

### Example 6

The flowability of Sokalan® HP 50 with an addition of 2% of Sipernat D 17 was investigated using 6 glass efflux vessels each having different efflux opening diameters. To this end, the vessels were each filled with the same amount of the product, the efflux opening was opened and flowability was assessed visually. Sokalan® HP 50 without the additive was tested as reference.

TABLE 6
flowability of Sokalan ® HP 50 with and without addition of 2% of Sipernat ® D 17

| Sokalan ® HP 50 | Assessment of flowability | | | | | |
|---|---|---|---|---|---|---|
| Glass efflux | 1 | 2 | 3 | 4 | 5 | 6 |
| vessel No. | | | | | | |
| Efflux opening | 2.5 | 5 | 8 | 12 | 18 | 24 |
| (mm) | | | | | | |
| a) no additive | not | not | not | not | not | not |
| | flowable | flowable | flowable | flowable | flowable | flowable |
| b) with 2.0% of | flowable, | flowable, | flowable, | flowable, | flowable, | flowable, |
| Sipernat ® D 17 | vessel | vessel | vessel | vessel | vessel | vessel |
| | has | has | has | has | has | has |
| | emptied | emptied | emptied | emptied | emptied | emptied |

List of Tradenames Used:

| | | |
|---|---|---|
| Sokalan ® HP 50 | (BASF) | polyvinylpyrrolidone, MM = 40 000 |
| | | g/mol, 96% pure powder |
| Sokalan ® HP 56 | (BASF) | copolymer based on vinylpyrrolidone and |
| | | vinylimidazole, MM = 70 000 g/mol, 97% |
| | | pure granules |
| Sipernat ® D 10 | (Evonik) | hydrophobically modified precipitated |
| | | silica |
| Sipernat ® D 17 | (Evonik) | hydrophobically modified precipitated |
| | | silica |

We claim:

1. A mixture comprising dye transfer inhibiting polymer and additive.

2. The mixture according to claim 1 wherein the dye transfer inhibiting polymer is in powder, granule or pellet form.

3. The mixture according to claim 1 or 2, wherein the additive is finely divided.

4. The mixture according to claims 1 to 3, wherein the dye transfer inhibiting polymer is selected from the group of homo- or copolymers of N-vinylpyrrolidone, N-vinylimidazole, N-vinylcaprolactam, 4-vinylpyridine, 4-vinylpyridine, diallyldimenthylammonium chloride, N-vinylformamide, N-vinylacetamide, vinylamine, allylamine, acrylamide and N-substituted acrylamides and wherein the nitrogen atoms are optionally derivatized.

5. The mixture according to any one of claims 1 to 4, wherein the dye transfer inhibiting polymer is polyvinylpyrrolidone, a vinylpyrrolidone-vinylimidazole copolymer, poly(4-vinylpyridine N-oxide) or poly(4-vinylpyridine N-carboxymethylbetaine).

6. The mixture according to any one of claims 1 to 3, wherein the dye transfer inhibiting polymer is a polycationic condensate which is obtained by reaction of at least one of the agents piperazine, 1-alkylpiperazine, 1,4-dialkylpiperazine, 1,4-bis(3-aminopropyl)piperazine, 1-(2-aminoethyl)piperazine, 1-(2-hydroxyalkyl)piperazine, imidazole, alkylimidazole with at least one second agent alkylene dihalides, epihalohydrin and/or bisepoxides and which may optionally be derivatized with methyl chloride, dimethyl sulfate, chloracetic acid, propanesultone or with hydrogen peroxide.

7. The mixture according to any one of claims 1 to 6, consisting of 90.0% -99.99% by weight of a dye transfer inhibiting polymer and 0.01% -10.0% by weight of an additive.

8. The mixture according to any one of claims 1 to 7, wherein the additive is a silica having a specific surface area of 30-500 m²/g.

9. The mixture according to any one of claims 1 to 8, wherein the additive is a silica having a tamped density of 50-300 g/L.

10. The mixture according to any one of claims 1 to 9, wherein the additive is the Ca²⁺ or Mg²⁺ salt of a C₁₆-C₂₂ fatty acid.

11. The use of a mixture according to any one of claims 1 to 10 in the manufacture of laundry detergents and cleaners.

12. The use of a mixture according to any one of claims 1 to 10 in the manufacture of textile auxiliaries.

13. The use of a mixture according to any one of claims 1 to 10 in the manufacture of cosmetic preparations.
